(19) United States
(12) Patent Application Publication
(10) Pub. No.: US 2003/0229909 A1
(43) Pub. Date: Dec. 11, 2003

(54) Cloning cats by nuclear transplantation

(75) Inventors: Mark E. Westhusin, College Station, TX (US); TaeYoung Shin, College Station, TX (US); Jane Pryor, Madisonville, TX (US); James Rugila, College Station, TX (US); D. C. Kraemer, College Station, TX (US)

Correspondence Address: FULBRIGHT & JAWORSKI L.L.P., 600 CONGRESS AVE., SUITE 2400, AUSTIN, TX, 78701, US

(73) Assignee: The Texas A&M University System

(21) Appl. No.: 10/366,068

(22) Filed: Feb. 13, 2003

Related U.S. Application Data
(60) Provisional application No. 60/356,626, filed on Feb. 13, 2002.

(51) Int. Cl. A01K 67/027

(52) U.S. Cl. 800/14; 800/21

(57) ABSTRACT

This invention is the first cat cloned by nuclear transfer, as well as any subsequent progeny produced by regular sexual reproduction. Nuclear transfer methods and techniques involving feline somatic cells, nuclei, or nuclear DNA, and the products thereof, are disclosed. In a single experiment 3 cloned embryos derived from the cumulus cell line and 2 embryos derived from fibroblast cells were transferred into a recipient female cat. Following embryo transfer after the period of gestation, a kitten was delivered. The cloned kitten has the exact DNA fingerprint as the cumulus cell line of the adult female cat from which the cumulus cell line was derived.

### BACKGROUND OF THE INVENTION

The present application claims priority to co-pending U.S. Provisional Application, Serial No. 60/356,626 filed Feb. 13, 2002. The entire text of the above-referenced disclosure is specifically incorporated herein by reference without disclaimer.

### FIELD OF THE INVENTION

The present invention relates generally to the fields of molecular biology, embryology and cloning. More particularly, it concerns techniques for and the animals produced by the cloning of felines, including domestic cats.

### DESCRIPTION OF RELATED ART

Animal cloning is still very inefficient and on average less than 10% of the cloned embryos transferred result in a normal live offspring. Even so, successful cloning of a variety of different species by a number of different laboratory groups has spawned tremendous interest in reproducing (cloning) specific animals. Many of these include animals that are genetically engineered. In other cases there is a significant demand for cloning specific animals due to their perceived genetic value. These include prized livestock, rare or endangered species, and pets. While there is no evidence that suggests cloning will be limited to only a few species, the prospect of cloning other species is often limited by a lack of understanding of the reproductive processes in those species or due to species-specific obstacles.

Man's interest in using nuclear transplantation as a tool for cloning animals dates back to the early 1900s (Spemann, 1938; Mclaren, 2000). In its simplest form, nuclear transplantation involves the transfer of a nucleus from one cell to another. For cloning animals this entails transferring the nucleus of a cell obtained from the individual to be cloned into an unfertilized ovum that has had its metaphase chromosomes removed. Once this occurs, the transferred nucleus is reprogrammed, and utilized by the egg cytoplasm so to direct development of a new embryo. This embryo is genetically identical to the animal from which the original donor cell was obtained and can be transferred into a surrogate female for gestation to term and birth of a clone.

Despite the long history and extensive research in this area, it was not until 1997 that the successful cloning of an adult animal was reported (Wilmut et al., 1997). Nuclei of cultured mammary epithelial cells derived from an adult ewe were transferred into enucleated sheep ova. The resulting cloned embryos were transferred into recipient ewes ultimately resulting in the birth of a single cloned lamb, Dolly. Since Dolly's birth, an explosion in research efforts targeted at cloning mammals has occurred, and cloned animals have now been reported for mice, cattle, goats, sheep and pigs (Wakayama et al., 1998, Wakayama and Yanagimachi, 1999; Cibelli et al., 1998; Kato et al., 1998; Wells et al., 1998; Kubota et al., 2000; Hill et al., 2000; Baguisi et al., 1999; Keefer et al., 2000; Wilmut et al., 1997;Campbell et al., 1996; Polejaeva et al., 2000; Onishi et al., 2000).

Cloning animals by nuclear transplantation requires the completion of several key steps: 1) acquisition of mature ova, 2) removal of metaphase chromosomes contained within the ova (enucleation), 3) transfer of cell nuclei from the animal to be cloned into the enucleated ova, 4) oocyte activation so to initiate embryonic development, and 5) transfer of the cloned embryos into surrogate mothers. The techniques/skills required to accomplish these steps differ between species. In addition, the efficiency of each step varies significantly, affecting the ease of which a particular animal can be cloned.

Cloned sheep, cattle, goats, pigs, and mice have now been produced by nuclear transplantation using somatic cells obtained from adult animals (WO9937143A2, EP930009A1, WO9934669A1, WO9901164A1, U.S. Pat. No. 5,945,577). Clones derived from adult cells of other species have not yet been reported. While, there is no evidence that suggests cloning will be limited to only a few species, the prospect of cloning other species is often limited by a lack of understanding of the reproductive processes in those species or due to species specific obstacles.

Cats represent a significant challenge when compared to other species that have been cloned, in part due to their unique reproductive physiology. While there are a few reports describing research involving nuclear transfer in cats (Fahrudin et al., 2001a; Skrzyszowska et al., 2001; Fahrudin et al., 2001b), live cloned offspring have yet to be reported. Current applications for cloning cats include genotype replication of pets, conservation of valuable genetics, conservation of endangered species and the creation of models for animal and human disease.

In combination with genetic engineering, cloning cats may also someday be used to produce a wide variety of new and useful animals with genotypic variations affording phenotypic traits such as increased longevity and allergen-free, among others. Cats are also important disease models and research tools. Cats are susceptible to many zoonotic pathogens that infect humans as well as being susceptible to many cancers and hereditary and congenital diseases that mimic the pathologies observed in analogous diseases in humans.

### SUMMARY OF THE INVENTION

The instant invention sets forth techniques and methods for the cloning of a feline based upon nuclear transplantation. The instant invention overcomes the noted obstacles in the art and provides a means to exactly replicate and transfer the genome of an organism from a somatic cell to produce an animal having an identical nuclear DNA complement, i.e., a clone. While techniques are known for cloning in a small number of alternate species, the instant invention sets forth the techniques necessary to achieve cloning by nuclear transfer in felines, including domestic cats.

The instant invention sets forth methods for transferring a feline nuclear genome from a somatic cell into a recipient cell to produce felids (synonymous with “felines”) that are genetically identical to the somatic cell. This involves achieving replication of a feline nuclear genome in a cell from which the genome was not derived or did not originate. Methods for cloning a feline may include one or more of the following steps: freezing, culturing, or thawing oocytes (activated and not activated, mature or immature), cybrids (activated and not activated), and cybrids that have undergone cell division; inducing a female canine to ovulate; gathering mature oocytes from a female feline; gathering immature oocytes from a female feline and maturing the oocytes in vitro; transferring the gathered mature or immature oocytes from one location to another; incubating the oocytes in transfer medium; incubating collected mature or immature oocytes in maturation medium with or without additives; enucleating oocytes; incubating enucleated oocytes in medium with or without Mg²⁺ and/or Ca²⁺; maturing oocytes after enucleation; activating oocytes prior to fusion, which may or not be activated again after a cybrid is formed; synchronizing the female feline from which oocytes are collected with surrogate females; collecting, obtaining, or preparing a somatic cell from tissue from a feline, which may be fresh, cultured, or previously cryopreserved, to serve as the source of nuclear DNA for the cloned feline; contacting nuclear DNA with or without an intact cell (donor nuclear DNA) with a recipient oocyte; fusing the donor nuclear DNA with the recipient oocyte to form a cybrid; activating the cybrid, chemically, physically, or electrically; incubating the oocyte and donor nuclear DNA in fusion medium that may or may not have Mg²⁺ and/or Ca²⁺ prior to fusion; incubating the cybrid in medium to allow it to divide; transferring the cybrid immediately or after one or more division cycles to a surrogate female feline; activating the cybrid in a medium, that may or may not contain Mg²⁺ and/or Ca²⁺ prior to transfer into a surrogate female; preparing a surrogate female feline for pregnancy; synchronizing the estrous cycle of a female surrogate feline and the development of a cybrid to be transferred into the surrogate; allowing an embryo to develop; providing progesterone to the surrogate female during pregnancy; and delivering the resulting cloned feline. It is contemplated that any of the embodiments discussed herein may be combined or employed with other embodiments discussed herein.

Ooctyes are used as the recipient for donor somatic nuclear DNA. Oocytes may be obtained from female felines by a number of ways well known to those of skill in the art. The collected oocytes may be matured in vivo or in vitro. They may be in metaphase I or metaphase II when used in methods of the invention, though in some embodiments of the invention, oocytes are in metaphase II. Females may or may not be given hormones so as to produce oocytes for retrieval. In some embodiments, oocytes may be collected by a number of ways including by first extracting the whole reproductive tract from a female animal or by excising the ovary from the mesovarium tissue, and then incubating the tract or ovaries in a buffer prior to isolating the oocytes. In other examples, oocytes are collected from the animal directly. Because isolation of oocytes may occur outside of the laboratory setting, oocytes may be transferred from one geographic location to another. Thus, in still further embodiments, the collected oocytes or tissue is incubated in a transport medium, including PBS, maturation medium, or TL Hepes. Additionally, the oocytes or tissue may be transported at temperatures up to 40 degrees celsius (°C.), though in specific embodiments the temperature is between 15 and 37° C. It is contemplated that the oocyte may be frozen prior to or during transportation. In such cases, it will be incubated at temperatures below 0° C., including being frozen in liquid nitrogen and at temperatures of less than −80° C. or −70° C.

Oocytes may have been matured in vivo and collected or they may first be collected and then matured in vitro. Oocytes can be matured in vitro after they have been isolated, but prior to enucleation. Alternatively, it is contemplated that oocytes may be matured in vitro after enucleation. To achieve maturation, they may be incubated in maturation medium. Oocytes may be incubated in maturation medium for up to 1, 2, 3, 4, 5, 6, 12, 18, 24, 30, 36, 42, 48, 54, 60, 66, 72 hours or up to 7 days, particularly if additives that slow maturation are not included. In the presence of additives, they may incubated for days or weeks longer. Additives include compounds that may affect the maturation of the oocyte, such as gonadotrophins, progesterone, milrinone, or dbcAMP.

Subsequent or prior to maturation, in vivo or in vitro, oocytes are enucleated in some embodiments of the invention. This may be accomplished by a number of means including physical removal of the nucleus. In some embodiments, a glass pipette is employed to remove the nucleus. In further embodiments, the oocyte is incubated in a medium; in specific embodiments, the medium is enucleation medium, which is Hepes-buffered TCM 199 supplemented with BSA, cytochalasin B and Hoechst 33342.

The invention includes the use of somatic cells as nuclear DNA donors from which the clone is derived. Any somatic cell may be used, and it may be derived from an embryo, fetus, sexually immature feline, or adult feline. Furthermore, the cell may be a cell from any organ of the body. In specific embodiments of the invention, the somatic cell is an adult cell, while in others, the somatic cell is an adult cumulus cell or fibroblast. An “adult cell” refers to a cell that is diploid and terminally differentiated, as opposed to a stem cell, certain embryonic cells, or sex cells. Though it is clear the invention further covers the use of stem cells and embryonic cells. It is contemplated that an entire somatic cell may be used for fusing with an oocyte or the nucleus or DNA from a somatic cell may first be isolated prior to fusing with an oocyte. Thus, a “composition comprising nuclear DNA from a somatic cell” includes intact cells, isolated nuclei, or isolated nuclear DNA from a somatic cell.

In some embodiments, these methods include contacting an enucleated oocyte from a donor organism with a composition that includes nuclear DNA from a feline somatic cell, fusing the oocyte and the composition containing nuclear DNA from a somatic cell to form a cybrid, and transferring the cybrid into the reproductive tract of a female feline. “Contacting” refers to the coming together, touching, associating, or juxtapositioning of one or more objects, which in the context of the present invention includes cells, nuclei, and genetic material.

For the purpose of the instant invention, “cybrid” refers to a cellular unit or structure that is composed of all or part of an oocyte cell and the nuclear DNA of a somatic cell and that is capable of undergoing cell division. A cybrid is formed when a somatic cell and a nucleus, cell, or nuclear DNA become fused. The term “cybrid” encompasses the terms “fused oocyte,” “NT (nuclear transfer or transplantation) embryo,” and “cloned embryo.” It is contemplated that the nuclear DNA may be in a nucleus and/or that the nucleus is contained in a somatic cell. An “embryo” refers to the developmental stage following the first cleavage of a diploid zygotic cell and preceding the fetal stage. An “enucleated” oocyte refers to an oocyte lacking all or part of the nucleus, including nuclear DNA. “Somatic cell” refers to any cell that is not a sex cell; a sex cell is haploid, while a somatic cell is not haploid. This method facilitates the production in a surrogate mother of an offspring whose mitochondrial DNA is derived solely, mainly, or partly (heteroplasmic) from one cell while its genomic DNA is derived from another cell. The product of this process is, therefore, a viable feline, which is included in the invention. For the purpose of the instant invention, a “cloned cat” or “cloned feline” or “cloned felid” refers to an animal originally derived from the transfer of a nuclear genome from a feline or cat somatic cell into a recipient cell, as opposed to being derived from the union of two sex cells.

It is contemplated that “nuclear DNA” refers to that portion of the genome that is located in the nucleus, contrary to “mitochondrial DNA,” which refers to that portion of the genome located in the mitochondria of a cell. “Nuclear genome” similarly refers to that portion of the genome located in the nucleus, while mitochondrial genome refers to the DNA located in the mitochondria. The nuclear genome may contain heterologous DNA sequences, for example, transgenic DNA sequences. “Heterologous” sequences refers to nucleic acid that is derived from a different source, i.e., organism, than the remainder of the DNA or to DNA sequences found in a location in the nuclear genome where it is not usually found.

It is further contemplated that the composition may contain any aqueous material such as medium and/or cellular material, such as all or part of a nucleus, or all or part of a cell. In some embodiments, the oocyte and the nuclear DNA, nucleus containing nuclear DNA, or somatic cell containing a nucleus containing nuclear DNA are from the same feline species and in a further embodiment, the oocyte and nucleus are from the same breed, though it is contemplated that methods do not require the same species or breeds. Thus, a cat oocyte may be used as the recipient for a somatic cell from a different feline breed or species. The present invention also includes methods and products of the methods in which an oocyte from a female is used for nuclear transfer involving a somatic cell from the same female animal. Throughout this application, the phrase “nucleus/cell” is intended to refer to a nucleus and/or a cell. It is further contemplated that either nuclear genomic material or a nucleus containing nuclear genomic material may be used interchangeably instead of a somatic cell comprising a nucleus in any of the methods described herein.

In some embodiments of the invention, a nucleus is isolated from a cell, for example, by physical manipulation, before coming in contact with an enucleated oocyte. A person of ordinary skill would recognize a variety of means of inserting or injecting a nucleus or the nuclear material from a cell into an enucleated oocyte. Alternatively, a cell containing a nucleus may be fused with an enucleated oocyte. In some embodiments of the instant invention, the oocyte and the nucleus/cell are fused by applying an electrical pulse. In specific embodiments of the invention, incubating the oocyte and the nucleus/cell in fusion media may further facilitate fusion.

In still further embodiments, the enucleated oocyte and/or somatic donor cell may be incubated in medium for up to 1, 2, 3, 4, 5, 6, 12, 24, 36, or 48 hours prior to fusion and/or activation. In some embodiments, the medium lacks Ca²⁺ or Mg²⁺, or both. This medium may be TCM199 with or without BSA, or it may be PBS, with or without BSA, that lacks Ca²⁺. It is also contemplated that they may be incubated in yet another medium thereafter, but prior to fusion and/or activation, for up to 1, 2, 3, 4, 5, 6, 12, 18, 24, 30, 36, 42, or 48 hours. If the oocytes/cells are incubated in the PBS, for example, they may then be incubated in TCM199.

In other embodiments, the methods further comprise a step of activating in vitro either the cybrid or the oocyte before it is contacted with a composition containing nuclear DNA. The oocyte may activated prior to fusion or prior to contacting with the composition comprising nuclear DNA, or it may be activated upon fusion. The cybrid may be activated upon formation or after fusion. Thus, it is contemplated that oocytes may be activated at least once prior to fusion, cybrids may be activated at least once upon or after fusion, and/or activations may occur in both situations. Oocytes may be activated up to 6, 12, 18, 24 or more hours or up to 1, 2, 3, 4, 5, 6, 7 or more days after retrieval, before or after maturation, before or after enucleation, or before fusion. Cybrids may be activated upon fusion or within 1, 2, 3, 4, 5, 6, 7, 8, 9, 10, 11, 12, 13, 14, 15, 16, 17, 18, 19, 20, 21, 22, 23, 24 or more hours after fusion. Activation may be achieved through the application of an electrical pulse, through the administration of a chemical compound, or by physical manipulation. Furthermore, a method of producing a cloned animal may involve more than one activation. An oocyte and/or the resulting cybrid may be activated 1, 2, 3, 4, 5, 6, 7, 8, 9, 10 or more times during the cloning process. In specific embodiments, the oocyte is not activated and the cybrid is not activated upon fusion.

In some embodiments of the present invention, the cybrid is transferred into the reproductive tract of a female cat immediately or at least before cell division occurs. While in other embodiments, the cybrid is incubated under conditions to promote cell division. The cybrid may undergo 1, 2, 3, 4, 5, 6, 7, 8, 9, 10, or more cell divisions, up to and including the morula or blastocyst stage before being transferred. For example, the cybrid may undergo at least two cell divisions before it is placed in a female surrogate. Meanwhile, the cybrid may be placed in medium, such as Tyrode's solution supplemented with additives. Additives include pyruvate, L-glutamine, calcium lactate, non-essential amino acids, and/or BSA. In some embodiments, the activated cybrid is placed in an in vitro culture medium, examples of which are well known to those of skill in the art, for up to 1, 2, 3, 4, or 5 days prior to transfer. In some embodiments, it is incubated in IVC 1 medium. It may be incubated in another in vitro culture media thereafter for up to 1, 2, 3, 4, or 5 days. The activated cybrid, in additional embodiments, is also placed in IVC 2 medium thereafter. It is contemplated that the amount of time total that the activated cybrid is kept in culture prior to transfer is 1, 2, 3, 4, 5, 6, 7, 8, 9, 10, 11, 12, 13, or 14 days, though in specific embodiments, the cybrid is transferred before 8 days in culture.

The synchronization of estrous of the surrogate female and embryo development of the cybrid is an aspect of some embodiments of the instant invention. In some aspects of the claimed invention, the estrous cycle of the surrogate female cat in which the cybrid is transferred is synchronized with the estrous cycle of the oocyte donor female. Two females may be considered synchronized if the oocyte donated by one female is capable of being fused with a nucleus/cell and successfully transferred and implanted in a second female. Thus, it is contemplated that there is a time window that encompasses both females, and that such window may be 1, 2, 3, or 4 days. In some embodiments, the surrogate female is induced to ovulate, manually, naturally, or through the use of hormones, on a particular day and the cybrid is transferred within 1, 2, 3, or 4 days of the expected or determined ovulation date, which is the date that is operative for synchronization. In still further embodiments, the cybrid that is transferred is at a corresponding stage of development as the surrogate female (with respect to her ovulation date); for example, if the surrogate is expected to ovulate on day 1, then the cybrid is activated within 48 hours (before or after) that day to synchronize its development with the surrogate. In further embodiments, the surrogate is at day 2 (one day after expected ovulation) and the cybrid that is transferred is also at day 2 (one day after activating) or was activated at least 24 hours before then. It is contemplated that synchrony includes when the stages of development between the female surrogate and the activated cybrid are within 6, 12, 24, 30, 36, 42, 48, 54, or 60 hours of each other. In specific embodiments, the stages of development are within a day (1-24 hours) of each other. In specific embodiments, synchrony is achieved when a female surrogate is expected to ovulate on the same day that a cybrid to be transferred to her is activated.

Generally, hormones, including human chorionic gonadotropin (hCG), progesterone, pregnant mare serum gonadotropin (PMSG), estrogen, follicle stimulating hormone (FSH), luteinizing hormone (LH), derivatives thereof, or a combination thereof, may be administered to females involved in the methods of the present invention. Females donating oocytes may be given hormones prior to oocyte retrieval. Surrogate females may be given hormones before or during pregnancy. In some embodiments, pregnant females are given progesterone after 30 days of gestation, such as at day 35 of gestation.

In the methods and compositions of the claimed invention, nuclear DNA may contain heterologous DNA sequences, such as a transgene. A transgene connotes a gene that has been transferred from one organism or species to another by genetic engineering.

The instant invention specifically contemplates the production of viable offspring from the creation of an embryo through nuclear transplantation. Therefore, embodiments of the instant invention encompass a feline whose nuclear genome is identical to a single parent, wherein the parent is multicellular (to distinguish mitosis). The feline animals of the present invention are cloned felines. A cloned feline will have cells whose nuclear genome is identical to, indistinguishable from, or derived from a single nuclear genome of a cell not obtained from the cloned feline itself (that is, obtained from another source). It may differ from naturally-occurring identical twins because some of its mitochondrial genome may be derived from a cell source that differs from the cell source of its nuclear genome. The mitochondrial genome of a cloned feline may be derived from one or more sources, including a recipient oocyte and/or the somatic cell from which the nuclear genome was derived.

The term “parent” is used to refer to a provider of nuclear genetic material. The word “single parent” refers to a sole provider of nuclear genetic material. The term “multicellular” indicates an entity composed of more than a single cell (that is, anything ≧2 cells), and thus, morulas, blastocyst, embryos, fetuses, and more developed forms of an organism are included. Therefore, the parent may be a morula, blastocyst, embryo, fetus, and up to and including an adult animal, which refers to an animal that has reached sexual maturity. An animal that is more developed than a fetus, but not yet an adult may also be a single parent, with respect to the offspring feline or cloned feline of the present invention.

It is specifically contemplated that methods of the invention concerning cats may be applied to other felines, and vice versa. It is further contemplated that any embodiments of the invention may be employed with any other embodiment of the invention.

Methods of obtaining such an offspring feline are disclosed herein. The cloned cats of the present invention may be produced using any of these methods. “Offspring” or “progeny” refer to any and all subsequent generations of an organism. “Immediate offspring” and “immediate progeny” mean the next generation (F1). The term “ancestor” refers to an organism that is of a predecessor generation (including parent, grandparent, great-grandparent, great-great grandparent, for example).

It is contemplated that animals produced by the disclosed methods will be reproductively viable. Therefore, an embodiment of the invention includes the immediate progeny produced by a mating between the feline produced by the disclosed methods and a second feline, as well as by subsequent matings involving any and all generations (progeny) of the immediate progeny.

The definitions are included in an effort to assist in the proper construction of terms used within the application. As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The technology described herein represents methods for and products of a strategy for cloning cats. The general approach involves nuclear transplantation where-by cells or cell nuclei derived from embryonic, fetal, juvenile, or adult cells (nucleus donor cells) are transplanted into enucleated oocytes. Oocytes containing the transplanted nucleus can be artificially activated before, after, or at the same time of fusion (contact, injection, etc.) so to initiate embryonic development. Oocytes may be artificially activated prior to, or after nuclear transplantation. The transplanted nuclei are reprogrammed to direct normal embryonic development. In the context of the instant invention, “reprogrammed nucleus” denotes a nucleus that is capable of directing embryogenesis and further embryonic development. The resulting cybrids can be transferred into surrogate females to produce cloned fetuses and offspring. The cybrids can also be used to produce chimeric embryos, fetuses and/or offspring. In addition, cybrids may be used to derive cells for another round of cloning. Nucleus donor cells may be cultured so to increase the number of cells available for nuclear transplantation, and frozen and thawed prior to use. Under the proper conditions, nucleus donor cells can be collected from deceased animals, cultured, frozen, thawed and used as nucleus donors for nuclear transplantation. Donor cells used for nuclear transplantation can be frozen and stored in liquid nitrogen for many years prior to thawing and using as nucleus donors for cloning by nuclear transplantation. For timing parameters described herein, the freezing of biological material will reset the timing parameters; for example, if a mature oocyte is frozen two days after it is retrieved, and thawed one year later, and kept in culture for one more day before it is fused, then that oocyte will be considered to have been fused 3 days after it was retrieved. Nucleus donor cells can also be genetically modified prior to utilization for nuclear transplantation therefore providing a method for producing genetically engineered animals. For example, DNA sequences may be added or deleted (e.g., knockout). Some of the techniques described herein have been described in U.S. Provisional Application Serial No. 09/882312 filed on Jun. 14, 2001, and U.S. Provisional Application Serial No. 60/347,424 filed on Jan. 10, 2002, both of which are herein incorporated by reference.

Methods of the invention include those described below and in the Examples. These methods may involve steps that are known to those of ordinary skill in the art, which can be found, for example, in Dresser et al., 1988; Bochenek et al., 2001; Davidson et al., 1986; Donoghue et al., 1992; Du et al., 2002; Fahrudin et al., 2001a; Fahrudin et al., 2001b; Farstad, 2000; Freistedt et al., 2001; Gomez et al., 2000; Gomez et al., 2001; Gomez et al., 2002; Goodrowe et al., 1988; Goodrowe et al., 1991; Guraya, 1965; Kanda et al., 1995; King et al., 2002; Kraemer et al., 1979; Lawler et al., 1993; Levy et al., 2001; Luvoni, 2000; Pope et al., 1997; Pope, 2000; Pope et al., 2000; Pope et al., 2002; Racchi et al., 1998; Skrzyszowska et al., 2001; Spindler et al., 2000; Swanson et al., 1996; Tsutsui et al., 2000; Verstegen et al., 1993; Wood et al., 1997 (all of which are incorporated herein by reference).

A. Collection of Oocytes and In Vitro/In Vivo Oocyte Maturation

It is currently believed that unfertilized oocytes matured to the metaphase II stage of meiosis are the most appropriate recipient oocytes for utilization in nuclear transfer to clone cats. However, there is some information to suggest that oocytes at others stages of meiosis may also serve as recipient oocytes for reprogramming somatic cell nuclei so to direct normal embryonic development of cloned cats. The following describes methods for obtaining oocytes from cats that can be used as recipient ova for nuclear transfer. In some embodiments, the oocytes are in metaphase II, though it is contemplated that oocytes in metaphase I may be employed. However, any unfertilized oocyte that has had its nucleus removed (see below) and is capable of reprogramming somatic cell nuclei and directing normal embryonic development may be used for cloning cats. These might include oocytes at various stages of meiosis and even include oocytes obtained from other animal species. Two methods may be employed for obtaining cat oocytes at the metaphase II stage of meiosis. These involve either in vitro maturation or in vivo maturation, discussed below.

Whether matured in vitro or in vivo, oocytes may be incubated in medium following collection. The oocytes may then be incubated at temperatures below freezing (in liquid nitrogen, around −80° C., or around −20° C., for example) or at temperatures of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C. They may be incubated for or not more than 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 102, 108, or 116 hours or more. During that time, the tissue may be transported to another location for further manipulation. At that time, the tissue may also be incubated in a transport medium, which includes PBS, maturation medium, or TL Hepes, or any other medium capable of supporting normal physiological conditions.

1. In Vitro Maturation

In vitro maturation has been implemented for obtaining bovine oocytes for use as recipient ova for nuclear transfer, as large numbers of unfertilized oocytes are obtainable using this method. It is contemplated that feline oocytes may also be matured in vitro. A variety of techniques may be employed to produce in vitro matured cat oocytes. Immature oocytes may be collected from female felines by a number of ways. Once collected, they may be incubated in a medium, with or without antibiotics. The medium may be any medium that supports their viability, including maturation medium. They may also be transported to another location for in vitro maturation.

The following represents one example of methods that may be employed. Cat ovaries are collected from local spay and neuter clinics and transported to the laboratory where immature oocytes are liberated from follicles. Follicular contents are then examined under a stereo microscope and the oocytes recovered and placed into fresh TL Hepes (Bavister, et al. 1983). Oocytes are then placed into culture wells containing 250 μl of maturation medium (composed of TCM 199 with Earle's salts supplemented with 0.36 mM pyruvate, 2.0 mM L-glutamine, 2.28 mM calcium lactate, 1.13 mM cysteine, 1% of a solution containing 10,000 U/ml Pennicillin G, 10,000 μg/ml Streptomycin (P/S), 10 ng/ml EGF, 1 IU/ml hCG, 0.5 IU/ml eCG and 3 mg/ml fatty acid free BSA (IVM medium)) for 24-30 hrs under 5% CO₂, 5% O₂, and 90%N₂ gas and humidified air atmosphere at 38° C. The time required for in vitro maturation may vary, but is normally approximately 24 to 96 hours. It is contemplated that oocytes may be matured in vitro for 1, 2, 3, 4, 5, 6, 7, 8, 9, 10, 11, 12, 13, 14, 15, 16, 17, 18, 19, 20, 21, 22, 23, 24, 36, 48, or more hours. It is further contemplated that oocytes may be matured in vitro in the presence of hormones such as human chorionic gonadotropin (hCG).

The oocytes may be cultured for 1, 2, 3, 4, 5, 6, 7, 8, 9, 10, 11, 12, 13, 14, 15, 16, 17, 18, 19, 20, 21, 22, 23, 24 hours or 1, 2, 3, 4, 5, 6, or 7 days. It is also contemplated that oocytes matured in vitro may be frozen, and that they may be cultured after they have been frozen.

2. In Vivo Maturation

Alternatively, cat oocytes that have been matured in vivo may be obtained and utilized as recipient oocytes for nuclear transfer to clone cats. The advantage of using these oocytes is they have undergone meiotic maturation under more natural conditions and therefore are expected to function more efficiently in terms of reprogramming cell nuclei and directing normal development of cloned cat embryos. The disadvantage of in vivo matured oocytes is they must be collected from live animals using surgical procedures. Also the number of in vivo matured oocytes that can be obtained for utilization for nuclear transfer is much more limited when compared to the number of oocytes available when employing methods for in vitro maturation. Methods for obtaining matured oocytes, including inducing superovulation, include administering to a female feline follicle stimulating hormone (FSH) or PMSG and then administering gonadotropin, such as hCG or LH, which are well known to those of skill in the art.

While the details presented here represent common methodology for obtaining oocytes at metaphase II of meiosis, other methods may also be used to obtain mature oocytes suitable as recipients for nuclear transplantation. Oocytes may be obtained from both live and deceased animals and utilized as recipient oocytes for nuclear transplantation. Where oocytes are obtained, they may be obtained at a stage prior to or during metaphase II. It is generally envisioned that oocytes may be cultured in vitro, however, in vivo culture is also specifically contemplated. In vivo culture means maturing the oocytes in vivo though it is contemplated that the oocytes may be cultured in the female from which they were obtained or in a different female. The oocytes may be cultured for 1, 2, 3, 4, 5, 6, 7, 8, 9, 10, 11, 12, 13, 14, 15, 16, 17, 18, 19, 20, 21, 22, 23, 24 hours or 1, 2, 3, 4, 5, 6, or 7 days. Oocytes also may be frozen; thus, such oocytes may be cultured before and/or after being frozen. Oocytes may be cultured for short or long periods of time. As mentioned above, oocytes from other species may also be used as recipient ova for nuclear transplantation.

B. Enucleation

Once an oocyte for nuclear transplantation is isolated, the nuclear material may be removed (enucleation). Methods of enucleation are well known in the art, such as described in Kanda et al., 1995 and U.S. Pat. No. 4,994,384 and 5,945,577, and EP0930009A1, which are incorporated by reference herein. For example, with felines, metaphase II oocytes are either placed in hepes-buffered TCM199, optionally containing 15 micrograms per milliliter cytochalasin B, for immediate enucleation, or may be placed in a suitable medium, for example, an embryo culture medium such as Tyrode's solution, plus 10% fetal bovine serum. Other media may be used, which would be known to those of skill in the art.

Prior to or after enucleation, the oocytes may be cultured. The time that the oocytes are cultured varies; they may be cultured for 6, 12, 18, 24 or more hours, or 1, 2, 3, 4, 5, 6, 7 or more days after being retrieved from ovaries or after being matured or enucleated. Enucleation may occur prior to being kept in culture as described above.

Enucleation accomplished microsurgically may involve the use of a micropipette to remove the polar body, the nuclear material, and the adjacent cytoplasm. The oocytes are then be screened to identify those of which have been successfully enucleated. Screening may be effected by staining the oocytes with 1 microgram per milliliter 33342 Hoechst dye in enucleation medium, and then viewing the oocytes under ultraviolet irradiation for less than 10 seconds. The oocytes that are successfully enucleated can then be placed in a suitable culture medium, e.g., Tyrode's solution plus 10% serum. Successful enucleation can be determined, for example, by confirming nuclear DNA has been removed.

Enucleation is traditionally carried out microsurgically, nevertheless, a person of ordinary skill would be aware that successful enucleation may also be achieved chemically, by centrifugation, by autoenucleation or by irradiation. See, for example Tatham, et al. (1995), Dominko, et al. (2000), Kamikova, et al. (1998), Elsheikh, et al. (1997), Fulka, et al. (1993).

The timing of enucleation may occur immediately after retrieval or after the oocyte reaches maturation, or it may occur about 1, 2, 3, 4, 5, 6, 7, 8, 9, 10, 11, 12, 13, 14, 15, 16, 17, 18, 19, 20, 21, 22, 23, 23, or 24 or more hours after collection or thawing. It is specifically contemplated that oocytes may be retrieved and enucleated prior to maturation, though in many embodiments, the oocyte is matured and then enucleated. While oocytes in metaphase II may be used, oocytes at other stages of meiosis, such as prophase I, metaphase I, anaphase I, telophase I, prophase II, anaphase II or telophase II, are contemplated.

C. Somatic Cell Sources

It is envisioned that the somatic cells, somatic cell nuclei or somatic cell nuclear DNA being transferred to the oocytes described above may be obtained from a variety of sources within the cat. Cell nuclei derived from embryonic, fetal, juvenile, or adult cells can be obtained from a variety of different tissue types including but not limited to cumulus, skin, oral mucosa, blood, bone marrow, lung, liver, kidney, muscle, and the reproductive tract. Each different cell type selected for use may require slight variations in the methods employed for culture, expansion, freezing, thawing, handling and treatment prior to utilization for cloning. However, the Examples provide a general approach that can be utilized for preparing cells obtained from cumulus tissue and skin tissue and provides an example of the basic methodology. It is contemplated that any cell, including dead cells, may be employed so long as the nuclear DNA is intact and can be reprogrammed to result in a cloned animal as described herein.

Because cloning an organism from an adult somatic cell may present issues related to aging, technology directed at addressing this are pertinent. This technology represents a strategy to minimize the continuous proliferation (cell division) that results from placing tissues and cells into culture. It is described in a U.S. Provisional Patent Application, Serial No. 60/211,862, entitled “Cryopreservation of Tissues for Use in Nuclear Transfer” and filed on Jun. 14, 2000 by the following inventors: Robert C. Burghardt, Mark Westhusin, and Dana Dean (referred to as “Burghardt et al., 2000”) and in nonprovisional patent application Serial No. 09/882,474. These applications are herein incorporated by reference in its entirety. The purpose of this technology is to minimize aging of cells that are used for somatic cell nuclear transfer into mature enucleated oocytes for the purpose of cloning or transgenic animal production. An additional purpose of the technology is to provide a substantial economic benefit by limiting the labor, supplies costs and storage costs associated with generating large numbers of cells prior to the time when cells are needed for nuclear transfer. With this technology it should be possible to generate the relatively small number of cells needed for nuclear transfer while minimizing the amount of cell division. This technology has also been utilized to recover cells post mortem from animals for use in nuclear transfer. Success has been achieved in recovering viable cells from a biopsy collected from a suitably refrigerated animal 96 hr post mortem.

Most cell types have a limited lifespan in culture that is characterized by the number of cell divisions or population doublings. It is thought that the lifespan may be limited by loss of pieces of DNA called telomeres at the ends of chromosomes because there is a correlation between the aging process and telomere length. Telomeres are the DNA-protein complexes that form the ends of eukaryotic linear chromosomes. These complexes maintain the integrity of genomic DNA by preventing degradation and fusion of chromosome ends and by ensuring complete chromosomal replication. The chromosomal ends are eroded each time the cell replicates its DNA during the process of cell division. Telomere-based models of cellular senescence would predict that nuclear transfer-derived animals would reach a critical telomere length earlier than a normal animal of the same age. Therefore methods to minimize cell division in cells that will be used for the purpose of somatic cell nuclear transfer would minimize telomere loss.

Cells for somatic cell nuclear transfer may be derived from surgical or biopsy specimens. One technique is routinely used to generate cell lines from surgical or biopsy samples by harvesting cells that grow from small pieces of tissue. The other techniques are designed to minimize the growth of cells until such time as cells are needed for the nuclear transfer. These cells can be derived by causing the outgrowth of cells from tissue pieces that have been suitably cryopreserved. There are essentially three steps taken to cryopreserve cells and tissues for somatic cell nuclear transfer. These techniques have been successfully applied to the collection of cells from healthy animals and from animals that have been dead for periods ranging from hours to days. In some examples, somatic cells for nuclear transfer are isolated after 1, 2, 3, 4, 5, 6, 7, 8, 9, 10, 11, 12, 13, 14, 15, 16, 17, 18, 19, 20, or more passages. The cells are frozen, then thawed, and cultured 1, 2, 3, 4, 5, 6, 7, 8, 9, 10, or more days prior to being used as nuclear donors for nuclear transfer.

D. Nuclear Transfer/Activation/In vitro Culture

Once enucleated, the oocytes are ready for implantation of alternate nuclear material by nuclear transplantation. Nuclear transfer involves the transplantation of viable nuclei from typically somatic cells to enucleated eggs. While the nuclei are generally derived from living cells, it is specifically contemplated, as described above, that nuclei derived from non-living tissue may also be successfully transplanted. The process constitutes isolating the recipient oocytes from a donor, enucleating the oocytes, transferring the desired cell nucleus into the enucleated oocyte, e.g., by cell fusion or microinjection, to effectively transfer the nucleus and form a cybrid. Techniques include those described herein as well as those known to those of skill in the art.

Prior to fusion or activation, the enucleated oocyte and/or somatic donor cell may be incubated in medium for up to 1, 2, 3, 4, 5, 6, 12, 24, 36, 48, 54, 60, 66, 72 or more hours prior to fusion and/or activation. In some embodiments, the medium may lack Ca²⁺ or Mg²⁺, or both. This medium may be TCM199 with or without BSA, or it may be PBS, with or without BSA, lacking Ca²⁺, or any other media that will not physiologically damage the cells, including those that lack Ca²⁺. If incubated in a medium that lacks Ca²⁺ or Mg²⁺, the cells may subsequently be incubated in another medium that has either Ca²⁺ or Mg²⁺ prior to activation. If the oocytes/cells are incubated in the PBS, they may then be incubated in TCM199 for up to 1, 2, 3, 4, 5, 6, 12, 18, 24, 30, 36, 42, 48, 54, 60, 66, 72, or more hours.

One method of fusion is electrofusion, nevertheless, fusion may alternatively be carried out by the exposure of the cells to fusion promoting chemicals, i.e. ethylene glycol, or the use of an inactivated virus such as, for example, Sendai virus. The term “fusion” or “fusing” as used herein refers to when the enucleated oocyte and donor nuclear material become a single entity. When an intact somatic cell is employed, fusion occurs when the somatic cell and the enucleated oocyte form a single cell membrane, effectively transferring the nucleus of the somatic cell into the oocyte. “Fusing” includes the injection of one compound into another. The cybrid may be transferred immediately to a synchronized surrogate female, or it may be cultured to at least the 2-cell developmental stage and then transferred into a surrogate for gestation. In the context of the instant invention, the nuclei are reprogrammed to direct the development of cloned embryos. The cloned embryos can also be combined with normal embryos, parthenogenetically-produced embryos or polyploid embryos (e.g., tetraploid embryos) to produce chimeric embryos, fetuses and/or offspring. Nuclear transfer techniques or nuclear transplantation techniques are known in the art, as set forth, for example in U.S. Pat. No. 5,945,577, Campbell et al., (1995); Collas et al., (1994); Keefer et a.l, (1994); Sims et a.l, (1993); WO 94/26884; WO 94/24274, and WO 90/03432, EP0930009A1 and U.S. Pat. No. 4,944,384 and U.S. Pat. No. 5,057,420.

In addition to the fusion of an oocyte and a nucleus/cell or nuclear DNA, the oocyte and/or the cybrid may be activated. Activation may occur at any one of the following times, though it is contemplated that activation may occur at more than one of the following times: the oocyte may be activated prior to contacting the source of the nucelar somatic DNA; the cybrid may be activated when it is created (upon fusion); the cybrid may be activated after fusion. “Activating” or “activation” is used according to its ordinary meaning in the art and refers to a physiological signaling (stimulus) that initiates embryonic development.

Activation of the oocyte may occur within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more hours of fusion or it may occur such hours after retrieval or maturation. Activation of the cybrid may occur within or after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more hours of fusion. Activation of the oocyte may occur within 1, 2, 3, 4, 5, 6, 7, 8, 9, 10 or more days after the oocyte is retrieved (or thawed) or before it is fused. The oocyte may be frozen before or after it is activated. If an oocyte is activated, activation may occur again after the cybrid is formed.

Activation may be achieved physically, electrically, or chemically. Physical activation includes pricking the oocyte or cybrid. Electrical activation involves applying at least one electrical pulse, though 1, 2, 4, 5, 6, 7, 8, 9, 10 or more pulsed are included. Chemical activation includes the use of ionomycin. Calcium in the medium promotes activation and may itself cause activation. Thus, when fusion occurs in the presence of calcium, activation may occur simultaneously with fusion. The oocyte or cybrid may be in fusion medium at the time of activation. After activation, oocytes or cybrids may be incubated in cycloheximide and/or cytochalasin B for 1, 2, 3, 4, 5, 6, 7, 8, 9, 10, or more hours between 37° C.-39° C. Cycloheximide concentrations include 1, 2, 3, 4, 5, 6, 7, 8, 9, 10, 11, 12, 13, 14, 15, 16, 17, 18, 19, or 20 μg/ml. Cytochalasin B concentrations include 0.5, 1, 2, 3, 4, 5, 6, 7, 8, 9, 10, 11, 12, 13, 14, 15, 16, 17, 18, 19, or 20 μg/ml. Incubation may be at temperatures of 20-40° C. In some embodiments, the incubation is done at 5% CO₂, 5% O₂, and 90% N₂ gas mixture in humidified air. They may be washed after that incubation step.

Furthermore, in some embodiments, the activated cybrid is then placed in an in vitro culture medium, such as IVC 1medium and/or IVC 2 medium for up to 1, 2, 3, 4, or 5 days prior to transfer. Though, in other embodiments transfer occur immediately after activation. As with the other media, any medium that supports the physiological integrity of the cell(s) may be employed in methods of the invention, and incubation conditions include O₂, CO₂, or N₂, or any combination thereof.

Following nuclear transplantation, the embryo may be allowed to undergo divisions and develop to the morula, and then blastocyst stage, or a later embryonic stage. In order to improve yield, the embryo may be split and the cells clonally expanded at any developmental stage. This manipulation facilitates the production of multiple embryos from a single successful nuclear transplantation. Clones from a cloned entity may be used as a source for additional cloning experiments.

E. Embryo Transfer

Some of the techniques of transfer and recipient animal management are generally standard procedures. Embryo transfer is a well known and widely used technology for the control of reproduction in agricultural animals. Embryo transfer and assisted reproduction technology is also well known in felines, including cats, as shown in references included herein. Traditionally females are superovulated and bred, the embryos recovered from the pregnant female and transferred to a surrogate mother. Alternatively, eggs are retrieved and mixed with sperm in a culture dish to allow fertilization. In the context of the instant invention, embryos (cybrid) are not derived from naturally fertilized ova but rather from enucleated ova that have been subjected to nuclear transplantation. The cybrid may be immediately transferred and/or implanted into a surrogate, or the transfer may occur after culturing. The cybrid may be transferred within 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours or within 1, 2, 3, 4, 5, 6, 7, or 8 days of fusion or activation. However, if the cybrid is frozen prior to transfer, it may be transferred days, weeks, months, or years, thereafter, though it may be transferred 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 hours or within 1, 2, 3, 4, 5, 6, 7, or 8 days of thawing. Where the cybrid is cultured, the cybrid may be cultured to the 2-, 4-, 8-, 16-, 32-, 64-, 128-, or 256-cell stage, or until the cybrid develops to a morula, or blastocyst. Due to failure of the embryo to implant in the reproductive tract or spontaneous abortion, embryo transfers generally have a less than 50% success rate. Early embryo development normally occurs in the Fallopian tube (oviduct), however, the Fallopian tube is more than a conduit between the ovary and the uterus. The Fallopian tube supplies a complex mixture of nutrients and may help to detoxify metabolic products produced by the embryo. Thus, it is important for embryos to successfully implant to be placed in a surrogate whose Fallopian tubes or uterus are capable of receiving and allowing implantation of the embryo. Whether a cybrid is transferred to the Fallopian tubes or uterus depends upon the developmental stage of the cybrid. Thus, depending upon the stage of cybrid development, synchronous transfers are important for successful implantation. For the purpose of the invention, a synchronous surrogate, is a female who is physically capable of being implanted with a cloned embryo and fostering embryo development. Synchrony may be achieved through selection (that is, choosing a female who is naturally at the appropriate stage of her estrus/estrous cycle), or a female may be induced to be in synchrony through the use of hormones, other pharmaceuticals, or through manual stimulation of the cervix such as the case with induced ovulators, which includes cats. A female surrogate, to induce her to ovulate, may be administered pregnant mare serum gonadotropin (PMSG) and/or human chorionic gonadotropin (hCG). Females may be given 50-500 IU of PMSG for 1, 2, 3, 4, 5, 6, 7, or more days. They may also be given hCG within 1-100 hours after administration of PMSG. 50-1000 IU of hCG may be given to a female feline at least once. Cybrids can be transferred into the females within 6, 12, 18, 24, 30, 36, 42, 48, 54, 60, 66, 72, 78, 84, 90, 96, 102, 108 or 116 hours or within 1, 2, 3, 4, 5, 6, 7, or 8 days hours following hCG administration or expected ovulation. Felines, as animals that are induced ovulators, may also be induced to ovulate manually according to techniques well known to those of skill in the art.

Furthermore, a surrogate female can be administered hormones throughout her pregnancy to assist in the maintenance of the pregnancy and the viability of the feline fetus. Progesterone may be administered to surrogate females. It may be given at dosages of 1, 2, 3, 4, 5, 6, 7, 8, 9, 10, 11, 12, 13, 14, 15, 16, 17, 18, 19, 20, 21, 22, 23, 24, or 25 mg for 1, 2, 3, 4, 5, 6, 7, 8, 9, 10, 11, 12, 13, or 14 times during pregnancy. It may be given daily or weekly as well. In some cases, 10 mg of progesterone is administered to a pregnant female weekly. It may administered as soon as the cybrid is transferred, or it may be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more days after transfer of the cybrid, although after day 35 of gestation is specifically contemplated.

F. Felines

The methods of the instant invention may be applied to any feline species, for example, felis catus or felis domesticus.

The felis catus or felis domesticus species has diverged into a number of breeds through selective breeding. The instant invention is nevertheless applicable to cats considered to belong to a recognized breed as well as cats considered of mix-breed. A list of exemplary breeds that may be cloned or the donors of cells or cellular material for use in the context of the instant invention includes, for example: Abyssinian, American Bobtail, American Curl, American Shorthair, American Wirehair, Angora, Asian Shorthair, Asian Semi-Longhair, Balinese, Bengal, Birman, Bombay, British Angora, British Shorthair, Burmese, Burmilla, California Spangled Cat, Chantilly/Tiffany, Chartreux, Chausie, Cherubim, Colorpoint Longhair, Colorpoint Shorthair, Cornish Rex, Cymric, Devon Rex, Egyptian Mau, Exotic Shorthair, Havana Brown, Himalayan, Honeybear, Japanese Bobtail, Javanese, Korat, LaPerm, Longhair, Maine Coon, Malayan, Manx, Munchkin, Nebelung, Norwegian Forest Cat, Ocicat, Ojos Azules, Oriental Longhair, Oriental Shorthair, Persian, Pixie Bob, Ragamuffin, Ragdoll, Russian Blue, Savannah, Scottish Fold, Selkirk Rex, Siamese, Siberian, Singapura, Snowshoe, Sokoke, Somali, Sphynx, Sterling, Tiffanie, Tiffany, Tonkinese, Traditional (Applehead) Siamese, Turkish Angora, Turkish Van, York Chocolate.

Other feline species include but is not limiting to: African golden cat (Profelis aurata), Andean mountain cat (Oreailurus jacobita), Asian golden cat (Catopuma temminckii), Black-footed cat (Felis nigripes), Bobcat (Lynx rufus), Bomean bay cat (Catopuma badia), Canadian lynx (Lynx canadensis), Caracal (Caracal caracal), Cheetah (Acinonyx jubatus), Chinese mountain cat (Felis bieti), Clouded leopard (Neofelis nebulosa), Cougar (Puma concolor), Eurasian Lynx (Lynx lynx), Fishing Cat (Prionailurus viverrinus), Flat-headed cat (Prionailurus planiceps), Geoffroys cat (Oncifelis geoffroyi), Iberian lynx (Lynx pardinus), Jaguar (Panthera onca), Jaguarundi (Herpailurus yagouarundi), Jungle Cat (Felis chaus), Kodkod (Oncifelis guigna), Leopard (Panthera pardus), Leopard cat (Prionailurus bengalensis), Lion (Panthera leo), Marbled cat (Pardofelis marmorata), Margay (Leopardus wiedii), Ocelot (Leopardus pardalis), Oncilla (Leopardus tigrinus), Pampas cat (Oncifelis colocolo), Pallas cat (Otocolobus manul), Rusty-spotted cat (Prionailurus rubiginosus), Sand cat (Felis margarita), Serval (Leptailurus serval), Snow leopard (Uncia uncia), Tiger (Panthera tigris), Wildcat (Felis silvestris).

G. Nucleic Acids

In the context of the instant invention, it may be important to screen developing embryos and postnatal animals for genetic identity to the donor organism. In the context of transgenic animals, the following techniques will be useful in screening for the presence of the transgene. This screening process may require the isolation, amplification and/or manipulation of genetic material. The following sections provide a brief overview of nucleic acids, their function and manipulation.

In the context of the instant invention, genes are sequences of DNA in an organism's genome encoding information that is converted into various products making up a whole cell. They are expressed by the process of transcription, which involves copying the sequence of DNA into RNA. Most genes encode information to make proteins, but some encode RNAs involved in other processes. If a gene encodes a protein, its transcription product is called mRNA (“messenger” RNA). After transcription in the nucleus (where DNA is located), the mRNA must be transported into the cytoplasm for the process of translation, which converts the code of the mRNA into a sequence of amino acids to form protein. In order to direct transport into the cytoplasm, the 3′ ends of mRNA molecules are post-transcriptionally modified by addition of several adenylate residues to form the “polyA” tail. This characteristic modification distinguishes gene expression products destined to make protein from other molecules in the cell, and thereby provides one means for detecting and monitoring the gene expression activities of a cell.

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

Screening methods of cells and/or organisms for the determination of effective nuclear transplantation may be carried out by hybridization screening, PCR, RFLP, Northern Blotting, Southern Blotting or other methods that a person of ordinary skill would view as adequate to determine nucleic acid identity. A brief description of exemplary methods follows.

1. Hybridization

Hybridization is understood to mean the forming of a double stranded molecule and/or a molecule with partial double stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and/or ionic strength of a desired stringency are determined in part by the length of the particular probe, the length and/or base content of the target sequences, and/or to the presence of formamide, tetramethylammonium chloride and/or other solvents in the hybridization mixture. It is also understood that these ranges are mentioned by way of example only, and/or that the desired stringency for a particular hybridization reaction is often determined empirically by comparison to positive and/or negative controls.

Accordingly, the nucleotide sequences of the disclosure may be used for their ability to selectively form duplex molecules with complementary stretches of genes and/or RNA. Depending on the application envisioned, it is preferred to employ varying conditions of hybridization to achieve varying degrees of selectivity of probe towards target sequence.

Nucleic acid molecules having sequence regions consisting of contiguous nucleotide stretches of about 13, 14, 15, 16, 17, 18, 20, 25, 30, 35, 40, 45, 50, 55, 60, 70, 80, 90, 100, 125, 150, 175, 200, 250, 300, 350, 400 or more basepairs (bp) to about 5000 bp, or even up to and including sequences of about 30-50 cM or so, identical or complementary to the target DNA sequence, are particularly contemplated as hybridization probes for use in embodiments of the instant invention. It is contemplated that long contiguous sequence regions may be utilized including those sequences comprising about 100, 200, 300, 400, 500, 600, 700, 800, 900, 1000, 1100, 1200, 1300, 1400, 1500, 2000, 2500, 3000, 3500, 4000, 4500, 5000, 5500, 6000, 6500, 7000, 7500, 8000, 8500, 9000, 9500, 10,000 or more contiguous nucleotides or up to and including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cM.

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

For applications requiring high selectivity, it is preferred to employ relatively stringent conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe and/or the template and/or target strand, and/or would be particularly suitable for isolating specific genes and/or detecting specific mRNA transcripts. It is generally appreciated that conditions may be rendered more stringent by the addition of increasing amounts of formamide.

2. Polymerase Chain Reaction

The technique of “polymerase chain reaction,” or “PCR,” as used herein generally refers to a procedure wherein minute amounts of a specific piece of nucleic acid, RNA and/or DNA, are amplified as described in U.S. Pat. No. 4,683,195; U.S. Pat. No. 4,683,202; and U.S. Pat. No. 683,194, which are herein expressly incorporated by reference. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid that is complementary to a particular nucleic acid.

3. Northern and Southern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting of RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

4. Restriction Fragment Length Polymorphism

“Restriction Enzyme Digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Incubation of about 1 hour at 37° C. is ordinarily used, but may vary in accordance with the supplier's instructions.

Restriction fragment length polymorphism (RFLP) analysis capitalizes on the selectivity of restriction enzymes to detect the genetic changes in specific loci. RFLP are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., Eco RI. Variations in RFLPs result from nucleotide base pair differences which alter the cleavage sites of the restriction endonucleases, yielding different sized fragments. Means for performing RFLP analyses are well known in the art.

As described in U.S. Pat. No. 5,580,729, herein expressly incorporated by reference, one means of testing for loss of an allele is by digesting the first and second DNA samples of the neoplastic and non-neoplastic tissues, respectively, with a restriction endonuclease. Restriction endonucleases are well known in the art. Because they cleave DNA at specific sequences, they can be used to form a discrete set of DNA fragments from each DNA sample. The restriction fragments of each DNA sample can be separated by any means known in the art. For example, an electrophoretic gel matrix can be employed, such as agarose or polyacrylamide, to electrophoretically separate fragments according to physical properties such as size. The restriction fragments can be hybridized to nucleic acid probes which detect restriction fragment length polymorphisms, as described above. Upon hybridization hybrid duplexes are formed which comprise at least a single strand of probe and a single strand of the corresponding restriction fragment. Various hybridization techniques are known in the art, including both liquid and solid phase techniques. One particularly useful method employs transferring the separated fragments from an electrophoretic gel matrix to a solid support such as nylon or filter paper so that the fragments retain the relative orientation which they had on the electrophoretic gel matrix. The hybrid duplexes can be detected by any means known in the art, for example, the hybrid duplexes can be detected by autoradiography if the nucleic acid probes have been radioactively labeled. Other labeling and detection means are known in the art and may be used in the practice of the present invention.

Nucleic acid probes which detect restriction fragment length polymorphisms for most non-acrocentric chromosome arms are available from the American Type Culture Collection, Rockville, Md. These are described in the NIH Repository of Human DNA Probes and Libraries, published in August, 1988. Methods of obtaining other probes which detect restriction fragment length polymorphisms are known in the art. The statistical information provided by using the complete set of probes which hybridizes to each of the non-acrocentric arms of the human genome is useful prognostically. Other subsets of this complete set can be used which also will provide useful prognostic information. Other subsets can be tested to see if their use leads to measures of the extent of genetic change which correlates with prognosis, as does the use of the complete set of alleles.

5. Mitochondrial DNA

In order to insure that effective nuclear transplantation has occurred, it may be necessary to screen both nuclear and cytoplasmic (mitochondrial) DNA. Mitochondria are the source of cytoplasmic genetic information. Mitochondria carry multiple copies of a circular genome that is replicated and expressed within the organelle and is inherited maternally. It is the only genetic element known to be inherited cytoplasmically from the maternal oocyte in mammals. Mitochondrial DNA in animals codes for 13 polypeptides that have been identified as components of the ATP synthesis system and codes for all of the transfer RNAs used in mitochondrial protein synthesis. The only non-coding portion of the genome is a region of approximately 900 base pairs that is referred to as the displacement loop or “D-loop”. This D-loop is involved in the control of transcription and replication of mitochondrial DNA. Variation in mitochondrial DNA will affect the size of the mitochondrial DNA population in the cell, abundance of mitochondrial DNA gene transcripts and translation products, and mitochondrial oxidative energy transduction capacity.

A variety of methods exist for isolating mitochondrial DNA, such as, for example, U.S. Pat. No. 5,292,639, Lindberg, et al., 1992 and incorporated herein by reference. Briefly, to isolate mitochondrial DNA from blood, leukocytes are isolated from anticoagulated blood by low-speed centrifugation after erythrocyte lysis with 140 mM ammonium chloride, pH 7.4. Leukocytes are lysed with 1% wt/vol Triton X-100 in the presence of 1% wt/vol sodium dodecyl sulfate. Nuclei and cell membranes are separated from the cytosolic fraction by centrifugation at 12,000 xg for 5 min. Soluble proteins are extracted from the resulting supernatant with 1:1 phenol:chloroform, and nucleic acids precipitated from the aqueous phase with 3 vol. ethanol. The procedure yields supercoiled and relaxed covalently closed and nicked circular mitochondrial DNA molecules, RNA and only trace amounts of nuclear DNA contaminants (U.S. Pat. No. 5,292,639).

Mitochondrial DNA may be manipulated and analyzed with the methods described in the previous sections as well as other techniques known to one of ordinary skill.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Nuclear Transplantation of Cybrids into Felines for the Production of

### Cloned Embryos

A. Material and Methods

Animal Care and Use. Domestic short or long hair female cats were used for this study. The cats were cared for in facilities and using procedures, which exceed the standards established by the American Association for Accreditation of Laboratory Animal Care (AAALAC). Cats were purchased from Class A dealers and quarantined for 2 weeks prior to integration into the colony. The cats were fed commercially prepared balanced diets and maintained individually in temperature controlled, environmentally enriched kennels with visual contact with other cats. They were exercised, mostly in groups, for at minimum one hour per day. Individualized, positive reinforcement training was provided.

Chemicals. Unless otherwise indicated, all chemicals were purchased from Sigma (St. Louis, Mo.).

Oocyte Recovery and In Vitro Oocyte Maturation (IVM). Reproductive tracts from normal queens greater than 6 months of age were collected by routine ovariohysterectomy at local veterinary clinics, placed immediately into physiological saline solution (PSS) at room temperature (22° C.-24° C.) and transported back to the laboratory for processing. Ovaries were removed from the tract and washed free from blood in TL-Hepes, and then repeatedly minced with a scalpel blade. With the aid of a stereomicroscope, class I & II oocytes (uniformly dark, densely and finely granulated ooplasm completely surrounded by multiple layers of cumulus cells) were selected. The immature cat ova were then cultured in TCM 199 with Earle's salts supplemented with 0.36 mM pyruvate, 2.0 mM L-glutamine, 2.28 mM calcium lactate, 1.13 mM cysteine, 1% of a solution containing 10,000 U/ml Penicillin G, 10,000 μg/ml Streptomycin (P/S), 10 ng/ml EGF, 1 IU/ml hCG, 0.5 IU/ml eCG and 3 mg/ml fatty acid free BSA (IVM medium) for 24-30 hrs under 5% CO₂, 5% O₂, and 90% N₂ gas and humidified air atmosphere at 38° C.

Enucleation. Following in vitro maturation, cumulus cells were removed from the ova by gently pipetting for 3 minutes in Hepes-buffered TCM 199 with Hank's salts supplemented with 0.1% hyluronidase. After removal of cumulus cells, the oocytes were washed and placed back into IVM medium as described above. For enucleation, denuded ova were placed for 20 minutes in Hepes-buffered TCM 199 supplemented with 3 mg/ml fatty acid free BSA, 15.0 μg/ml cytochalasin B and 5 μg/ml Hoechst 33342 (enucleation medium). All oocytes were carefully selected for the presence of a polar body and homogeneous cytoplasm. The ova were enucleated using a 18-20 μm (outside diameter) beveled glass pipette mounted on Narshige micromanipulators while viewing with a Zeiss Microscope. Enucleation consisted of removing both the polar body and metaphase chromosomes, and was confirmed by observation under UV light. Only enucleated ova were selected for use as recipient ova for nuclear transfer.

Cell culture and preparation of donor cells. Adult fibroblast cells were isolated from oral mucosa obtained from an adult male cat, and cultured in DMEM/F12 (Gibco), supplemented with 10% FBS for 3-5 days at 37° C. in an atmosphere of in 5% CO₂ and air. Cells were passaged 1-7 times. Cells were then collected, frozen and stored in liquid nitrogen (N₂). Three to 5 days prior to nuclear transfer the cell line was thawed and maintained in 4-well dishes (Nunc, Denmark) in DMEM/F12, supplemented with 10% FCS+1% P/S at 37° C. in an atmosphere of in 5% CO₂ and air. Cells were in culture until confluent.

Alternatively, a second cell line was derived from cumulus cells obtained from and adult female cat. Queens were given an intramuscular injection of 150-200 IU of pregnant mare serum gonadotropin (PMSG, Sigma-Aldrich, St. Louis) followed 75-84 hours later by an injection of human chorionic gonadotropin (hCG, Chorulon, Intervet Inc., Millsboro, De.). Unfertilized ova were surgically collected approximately 48 hours following the hCG injection by flushing the oviducts with TL Hepes. Ova were isolated with the aid of a stereomicroscope and cumulus cells were removed from the ova by gently pipetting for 3 minutes in Hepes-buffered TCM 199 with Hank's salts supplemented with 0.1% hyluronidase. Cumulus cells were then placed into DMEM/F12 medium, washed by centrifugation, and transferred into tissue culture wells containing DMEM/F12. The cells were cultured for 5 days at 37° C. in an atmosphere of 5% CO₂ and air until confluent.

Recombination. For recombination with the enucleated oocytes, the cultured donor cells were removed from the incubator and trypsinized using a 1% trypsin-EDTA solution (Gibco) for less than 1 min with gentle pipetting. The cell suspension was then diluted in 5 ml of Hepes-buffered TCM 199 supplemented with 10% FCS, and washed by centrifugation (3 min, 200 g). The cells were then resuspended in fresh Hepes-buffered TCM199 supplemented with 10% FCS.

Enucleated ova and donor cells were then placed into a petri dish containing either Hepes buffered TCM 199 supplemented with 0.3% BSA (treatment 1), or Ca²⁺, Mg²⁺ free D-PBS with 0.3% BSA (treatment 2). Nucleus donor cells were combined with enucleated oocytes using the same pipette that was employed for enucleation. The oocyte-fibroblast cybrids were then returned to TCM199 supplemented with 0.3% BSA at 38° C. in an atmosphere of 5% CO₂ and air, until electrofusion.

Electrofusion and post activation. Two separate treatments were employed for electrofusion and activation of the oocyte/cell cybrids.

For treatment 1, fusion and activation were performed simultaneously. In brief, the cybrids were equilibrated in 0.3 M mannitol solution containing 0.1 mM Ca²⁺ and 0.1 mM Mg²⁺, then transferred to an electrofusion chamber containing the same medium. Cell fusion was induced by applying 2, 3.0 kV/cm 25 μsec DC pulses delivered by a BTX Electrocell Manipulator 200 (BTX, San Diego, Calif.). The cybrids were then removed from the fusion chamber, washed and incubated in TCM 199 supplemented with 0.3% BSA and 5.0 μg/ml cytochalasin B, at 38° C. in and atmosphere of 5% CO₂ and air. After one hour, the cybrids were removed from the incubator to evaluate cell fusion. They were then transferred into TCM 199 supplemented with 0.3% BSA and returned to the incubator. One hour later they were transferred into TCM 199 supplemented with 0.3% BSA, 10 μg/ml cycloheximide and 5 μg/ml cytochalasin B; then cultured for 6-7 hours at 38° C., in a humidified atmosphere of 5% CO₂, 5% CO₂, 90% N₂.

For treatment 2, electrofusion was performed as described for treatment 1 except that the fusion medium consisted of 0.3 M mannitol solution containing 0.1 mM Mg₂₊, but without Ca²⁺. Following electrofusion the cybrids were treated as described in treatment one except a second electropulse was applied to induce activation. Two hours after electrofusion, fused cybrids were removed from the incubator and equilibrated in 0.3 mM mannitol containing 0.1 mM Ca²⁺ and 0.1 mM Mg²⁺, then placed into a fusion chamber containing the same medium and electropulsed by applying 2×, 1.0 KV/cm 50 μsec pulses, 5 seconds apart. The ova were then removed from the fusion chamber, washed, and incubated for 6-7 hrs in TCM 199 supplemented with 0.3% BSA, 10 μg/ml cycloheximide and 5 μg/ml cytochalasin B in a 5% CO₂, 5% O₂, 90% N₂ gas mixture in humidified air at 38° C.

Following the fusion/activation treatment, the fused cybrids were either transferred into synchronized recipient females for the production of offspring or placed into in vitro culture. With the successful clone, the fused/activated cybrids were placed in culture overnight and transferred the following day.

In vitro culture and embryo transfer. For in vitro culture, nuclear transfer embryos were placed in modified Tyrode's solution supplemented with 0.36 mM pyruvate, 1.0 mM L-glutamine, 2.28 mM calcium lactate, 1% non-essential amino acids (NEAA) and 3 mg/ml fatty acid free BSA (IVC 1 medium) for 1-3 days. After the initial culture period, embryos were switched to modified Tyrode's solution supplemented with 0.36 mM pyruvate, 1.0 mM L-glutamine, 2.28 mM calcium lactate, 2% MEM essential (EAA), 1% non-essential amino acids (NEAA) and 10% heat inactivated fetal bovine serum (Hyclone, Logan, Utah) (IVC 2 medium) and cultured for up to 7-8 days under 5% CO₂, 5% O₂, 90% N₂ gas mixture in humidified air at 38° C. Embryo development was assessed for cleavage on day 3 of in vitro culture and percentage of embryos developing to the blastocyst stage was recorded following 7 days of in vitro culture.

Synchronization of recipient females and embryo transfer. In some cases, cloned embryos were transferred into recipient queens for the production of cloned offspring. To synchronize the estrus cycle, female cats were given an intramuscular injection of 150-200 IU of pregnant mare serum gonadotropin (PMSG, Sigma-Aldrich, St. Louis) followed 75-84 hours later by an injection of 250-500 IU human chorionic gonadotropin (hCG, Chorulon, Intervet Inc., Millsboro, Del.). Nuclear transfer was performed on the day of expected ovulation, approximately 36-48 hours following the hCG injection.

For embryo transfer surgery was performed on recipient females to expose the reproductive tract. Cloned embryos were removed from culture, loaded into a embryo transfer pipette and transferred into either the oviduct or the uterus, depending on their age and stage of development relative to the estrus cycle stage of the recipient female. In some cases, cloned embryos were transferred into the oviduct the same day nuclear transfer was performed. In other cases the embryos were cultured in vitro several days prior to embryo transfer.

Recipient female treatment and care. Following embryo transfer, transabdominal ultrasonography was utilized to monitor for pregnancy. In some cases, animals determined to be pregnant were given supplemental progesterone in the form of 10 mg medroxyprogesterone acetate, per os—orally (PO), once a week beginning approximately day 35 of gestation with the last dose administered on day 56 of gestation. Following cessation of progesterone treatment, blood samples were collected and analyzed, daily, to monitor serum progesterone levels. Once parturition was initiated as indicated by a drop in progesterone and signs of labor, a cesarean section was performed to deliver the cloned offspring.

### EXAMPLE 2

### Nuclear Transplantation Using Feline Nucleus Donor Cells Derived From Oral

### Mucosa Of Adult Male Cat

Results of experiments involving nuclear transfer in cats are provided in Table 1 below. For treatment 1, 108 trials were performed resulting in 52 cloned cat embryos that were transferred into 3 synchronized recipient queens 1-3 days following nuclear transfer. No pregnancies were obtained. For treatment 2, 197 trials were performed to obtain 84 embryos, which were transferred into 8 recipients. One of these was diagnosed pregnant approximately 26 days following embryo transfer. Ultrasonography was used to monitor development of a single conceptus, which ceased to develop and was surgically removed at approximately 44 days of gestation. Subsequent DNA analysis confirmed the pregnancy was derived from a cloned embryo.

[image]

[image]

### EXAMPLE 3

### Nuclear Transplantation Using Feline Nucleus Donor Cells Derived from Cumulus Cells of Adult Female Cat

Due to the lack of success in the first experiment, a second cell line was derived from cumulus cells obtained from an adult female cat maintained in the cat colony, and utilized for nuclear transfer. In a single experiment, 3 cloned embryos derived from the cumulus cell line (details in Table 2) and 2 cloned embryos derived from fibroblast cells (Table 1, Recipient “Allie”) were transferred into a recipient queen. Pregnancy was detected by ultrasonograpy at 22 days of gestation and a kitten was delivered by C-section 66 days following embryo transfer. The kitten appeared completely normal and was vigorous at birth.

Five additional experimental replications were carried out that involved nuclear transfer using cumulus cells derived from an adult cat, Rainbow. In brief, this was simply a replication of the experimental design that resulted in the birth of the cloned kitten CC. These repetitions and the data are detailed in Table 2 below (Rainbow cumulus passage 3-5). No additional pregnancies were obtained from these trials.

TABLE 2
Nuclear transplantation (cloning) in cats using in vitro matured feline recipient ova and feline
nucleus donor cells derived from cumulus cells from an adult female cat - Rainbow

| Donor | No. | No. | No. | Fusion | | No. Embryo | | | Manipulation |
|---|---|---|---|---|---|---|---|---|---|
| (passage times) | Enucleated | Insertion | Fused | (%) | Recipient | Transfer | Pregnant | Treatment | media |
| Rainbow | 8 | 8 | 3 | 37.5 | Allie** | 3 | Positive, | II | D-PBS (−) |
| Cumulus | | | | | | | Kitten | | |
| (PI) | | | | | | | born | | |
| | | | | | | | Dec. 22, 2001 | | |
| Rainbow | 6 | 5 | 3 | 60.0 | Maple | 3 | No | II | D-PBS (−) |
| cumulus (P5) | | | | | | | | | |
| Rainbow | 14 | 12 | 4 | 33.3 | Bobbi | 4 | No | II | D-PBS (−) |
| cumulus (P4) | | | | | | | | | |
| Rainbow | 12 | 8 | 5 | 62.5 | Felina | 5 | No | II | D-PBS (−) |
| cumulus (P4) | | | | | | | | | |
| Rainbow | 25 | 18 | 8 | 44.4 | Freesbee | 8 | No | II | D-PBS (−) |
| cumulus (P5) | | | | | | | | | |
| Rainbow | 32 | 29 | 12 | 41.4 | Tara | 12 | No | II | D-PBS (−) |
| cumulus (P3) | | | | | | | | | |
| Total | | 97 | 80 | 35 | 6 | 35 | ⅙ | | |

### EXAMPLE 4

### Microsatellite Analysis of Cloned Feline

The kitten's coat color suggested it was derived from a cumulus cell. Therefore, DNA samples were obtained from: 1) the cumulus cell line used for nuclear transfer; 2) blood from the cat this cell line was derived; 3) blood from the surrogate mother; and an oral swab of the kitten. In addition, control feline samples allowed the comparison to a random bred cat genetic database. Analysis of seven, unlinked, highly polymorphic feline specific microsattelite loci confirmed the kitten was a clone. Analysis was performed on an ABI 377 using the analysis software STRand, developed by the UC Davis Veterinary Genetics Laboratory. By direct counting, the cloned kitten did not have the DNA profile as the surrogate mother nor any sample in the database but had the exact same DNA profile as the cumulus cell line and the cat from which this cell line was derived. As with other genetically identical animals exhibiting multiple coat colors, the cloned kitten's color pattern is not exactly the same as the cell donor. However, the coat color distribution is not exactly the same. The pattern of pigmentation in multi-colored animals is the result of genetic factors as well as developmental factors that are not controlled by genotype. Data for evaluation of the seven markers are shown in Table 3. The numbers represent the size of amplified mircosatellite markers. Statistical analyses of these data indicate that the probability of the specific microsatellite analysis of the cloned kitten matching another cat is 9×10⁻¹⁶.

TABLE 3
Analysis of feline genetic markers

| | Cumulus Cell | Cumulus Cell | Cloned | Surrogate |
|---|---|---|---|---|
| Feline | Donor | Line | Kitten | Queen |
| Markers | Blood | Cultured Cells | Cheek Cells | Blood |
| FCA229 | 164/164 | 164/164 | 164/164 | 166/166 |
| FCA290 | 222/222 | 222/222 | 222/222 | 212/218 |
| FCA305 | 1941196 | 194/196 | 194/196 | 196/196 |
| FCA441 | 165/169 | 165/169 | 165/169 | 165/169 |
| FCA078 | 196/198 | 196/198 | 196/198 | 194/200 |
| FCA201 | 159/163 | 159/163 | 159/163 | 143/159 |
| FCA224 | 154/160 | 154/160 | 154/160 | 160/162 |

Besides the utilization of cumulus cells as nucleus donors rather than fibroblasts, several other observations are worth noting that may have played a role in the outcome of these experiments. Both cloned conceptus derived from fibroblast cells and the cloned kitten derived from cumulus cells were the result of treatment 2, which involved several modifications to the nuclear transfer protocol. In an attempt to minimize oocyte activation at the time of electrofusion, calcium was not included in the fusion medium. In addition, all micromanipulation was carried out in DPBS without calcium. Finally, in addition to the electrical pulse given at the time of electrofusion, a second pulse was administered to induce oocyte activation, 2 hours after the initial electrofusion pulse.

Previous research involving other species has indicated that a major cause of developmental failure in clones is abnormal placentation. Given this information and the knowledge that during pregnancy in cats, progesterone is first produced by corpra lutea and later in gestation by the placenta, the recipient female that became pregnant following the transfer of a cloned embryo derived from cumulus cells, was given supplemental progesterone in the form of 10 mg medroxyprogesterone acetate, per os—orally (PO), once a week beginning approximately 30 or more days of gestation with the last dose administered on day 56 of gestation. Following cessation of progesterone treatment, blood samples were collected and analyzed, daily, to monitor serum progesterone levels. Once parturition was initiated as indicated by a drop in progesterone and signs of labor, a cesarean section was performed to deliver the cloned offspring.

### EXAMPLE 5

### Analysis of Genetic Markers Of Cloned Tundra Fetuses

Further trials involving nuclear transfer and utilizing adult fibroblast cells obtained from a Himilayan cat (Tundra) were conducted. One additional pregnancy was obtained that resulted in twins (Table 4). Both of the fetuses derived from this trial exhibited normal heart beats when examined by ultrasound at day 25 of gestation. Unfortunately, however, neither survived to term. One of the wins ceased to exhibit a normal heartbeat at D58 of gestation. However, there were no signs of intrauterine infection, a drop in progesterone levels or other problems, therefore the pregnancy was monitored on a daily basis by ultrasound. At D65, a C-section was performed. However, the heartbeat on the remaining live fetus had also ceased, therefore both kittens were still born. Tissue samples were taken from both fetuses and histological evaluations were performed. At the present time no specific cause of death has been documented nor any developmental abnormality identified as the cause of death of the cloned kitten fetuses. Table 5 represents the genetic marker analysis of cloned Tundra fetuses proving that the fetal twins were in fact clones of Tundra.

Table 6 provides a summary of the cats cloned using adult fibroblast and cats cloned using cumulus as a genetic donor. Of the cats cloned form cumulus cells, one kitten (cc) was born.

TABLE 4
Nuclear transfer with Adult fibroblast cells (Tundra)

| Donor | No. | No. | No. | Fusion | | No. Embryo | | | Manipulation |
|---|---|---|---|---|---|---|---|---|---|
| (passage times) | Enucleated | Insertion | Fused | (%) | Recipient | Transfer | Pregnant | Treatment | media |
| Tundra | 15 | 12 | 6 | 50.0 | Pez | 6 | No | II | D-PBS (−) |
| P2 (oral) | | | | | | | | | |
| Tundra | 41 | 25 | 12 | 48.0 | Maple | 12 | Positive | D-PBS (−) | |
| P2 (oral) | | | | | | | (Twins) | | |
| Tundra | 14 | 11 | 7 | 63.6 | Bobbi | 7 | No | II | D-PBS(−) |
| P5 (oral) | | | | | | | | | |

TABLE 5
Genetic Marker Analysis of cloned fetuses in Cat

| | Tundra | Cloned | Cloned | Maple |
|---|---|---|---|---|
| | (Genetic | fetus 1 | fetus 2 | (surrogate |
| Marker | donor) | (T1) | (T2) | queen) |
| FCA35 | 137/137 | 137/137 | 137/137 | 137/137 |
| FCA78 | 187/195 | 187/195 | 187/195 | 199/201 |
| FCA102 | 176/184 | 176/184 | 176/184 | N/A |
| FCA117 | 163/163 | 163/163 | 163/163 | 161/165 |
| FCA201 | 143/157 | 143/159* | 143/157 | 161/161 |
| FCA224 | 160/160 | 160/160 | 160/160 | 160/174 |
| FCA229 | 168/168 | 168/168 | 168/168 | 168/168 |
| FCA240 | 156/156 | 156/156 | 156/156 | 164/172 |
| FCA290 | 214/214 | N/A | 214/214 | 212/214 |
| FCA293 | 195/195 | 195/195 | 195/195 | 189/189 |
| FCA391 | 251/251 | 251/251 | 251/251 | 251/259 |
| FCA441 | 157/157 | 157/157 | 157/157 | 169/169 |
| FCA453 | 188/196 | 188/196 | 188/196 | 188/188 |
| FCA651 | 135/135 | 135/135 | 135/135 | 133/135 |
| FCA674 | 172/172 | 172/172 | 172/172 | 150/150 |

*There was one genotyping error. Locus 201 reads 159 for T2 while the donor and T1 are 157. It is HIGHLY LIKELY that this was a slight genotyping error. Note that 15 markers were used, double the number done for CC. Thus, the odds are virtually incalculable that the two fetuses are not clones.

TABLE 6
Summary of Cat Cloning with Adult Fibroblast vs. Cumulus as a Genetic Donor

| Donor | # | # | # | # | # | # |
|---|---|---|---|---|---|---|
| cell | enucleated | insertion | fused(%) | transferred | pregnant(%) | offspring(%) |
| Adult | 267 | 245 | 110(44.9) | 110 | 2/11 | 0 |
| fibroblast | | | | | (18.2) | |
| Cumulus | 97 | 80 | 35(43.8) | 35 | 1/6 | 1(3) |
| | | | | | (16.7) | |

All of the compositions and methods disclosed and claimed herein can be made and executed without undue experimentation in light of the present disclosure. While the compositions and methods of this invention have been described in terms of embodiments, it will be apparent to those of skill in the art that variations may be applied to the compositions and methods, and in the steps or in the sequence of steps of the methods, described herein without departing from the concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,994,384

U.S. Pat. No. 5,057,420

U.S. Pat. No. 5,292,639

U.S. Pat. No. 5,580,729

U.S. Pat. No. 5,945,577

U.S. Pat. No. 6,011,197

Baguisi, et al., Nat. Biotechnology, 17:456-461, 1999.

Bavister, et al., Biol. Reprod.., 28(1):235-47, 1983.

Bochenek, et al., Theriogenology, 55(1):256, 2001.

Campbell K H, et al., Nature, 380:64-66, 1996.

Cibelli J B, et al., Science, 280:1256-1258, 1998.

Collas et al, Mol Reprod Dev. 38(3):264-7, 1994.

Davidson, et al., Veterinary Radiol., 27(4):109-114, 1986.

Dominko, et al. Biol Reprod., 62(1): 150-4, 2000.

Donoghue, et al., Biol. Reproduction, 46:972-980, 1992.

Dresser, et al.,J. Experi. Zool., 246:180-186, 1988.

Du, et al., Theriogenology, 57(1):409, 2002.

EP0930009A1

Elsheikh, et al. J. Vet. Med. Sci., 59(2):107-13, 1997.

Fahrudin, et al., Theriogenology, 55:268(abstr), 2001 a.

Fahrudin, et al., Asian-Aust J Anim Sci., 14:1057-1061, 2001b.

Farstad, Animal Reprod. Sci., 60-61(375-387):358-369, 2000.

Freistedt, et al., Biol. Reproduction, 65:91-13, 2001.

Fulka et al. Mol. Reprod. Dev., 34(4): 427-30, 1993.

Fulka et al,. Int J Dev Biol., 37(3): 433-9, 1993.

Gomez, et al., Reprod. Fertil. Dev., 12:423-433, 2000.

Gomez, et al., Theriogenology, 55(1):472, 2001.

Gomez, et al., Theriogenology, 57(1):415, 2001.

Goodrowe, et al., Biol. Reproduction, 39:355-372, 1988.

Goodrowe, et al., Biol. Reproduction, 45:466-470, 1991.

Guraya, J. Exp. Zool., 160:123-136, 1965.

Hill et al., Biology of Reproduction, 62:1135- 1140, 2000.

Kanda, et al., J. Vet. Med. Sci., 57(4):641-646, 1995.

Kamikova, et al. Reprod. Nutr. Dev., 38(6):643-51 (1998.

Kato et al., Science 282:2095-2098, 1998.

Keefer et al, Biol Reprod, 50(4):935-9, 1994.

King et al., Theriogenology 57(1), 703, 2002.

Kitiyanant et al., Theriogenology 57(1), 423 (2002.

Kraemer et al, Theriogenology, 11:51-62, 1979.

Kubota C et al., Proc. Natl. Acad. Sci., 97:990-995, 2000.

Lawler, et al., J. Reprod. Fert. Suppl., 47:57-61, 1993.

Levy, et al., JAVMA, 219(10):1401-1405, 2001.

Lindberg, et al., Biochem. Genet., 30:27, 1992.

Luvoni, Reprod. Nutr. Dev., 40:505-512, 2000.

Mclaren A., Science, 228:1775-1780, 2000.

Onishi et al., Science, 289:1188-1190, 2000.

Polejaeva et al., Nature, 407:86-90, 2000.

Pope, Theriogenology, 53:163-174, 2000.

Pope et al., Journal of Reproduction and Fertility Supplement, 51:69-82, 1997.

Pope et al., Theriogenology, 57(1):464, 2002.

Racchi et al., Blood Cells, Molecules and Diseases, 24(21):439-447, 1998.

Skrzyszowska et al., Theriogenology, 55:292(abstr), 2001.

Sims et al., Proc. Natl. Acad. Sci., 90:6143-6147, 1993.

Spemann, Embryonic Development and Induction. 1938. New Haven, Conn.: Yale Univ Press. 210-211. Cited in, Di Beradino M A., Genomic potential of differentiated cells. New York: Columbia University Press 28-53, 1997.

Spindler, et al., Molec. Reprod. Dev., 56:163-171, 2000.

Swanson, et al., Molec. Reprod. Dev., 43:298-305, 1996.

Tatham, et al. Biol. Reprod, 53(5): 1088-94, 1995.

Tsutsui, et al., J. Vet. Med. Sci., 62(11):1169-1175, 2000.

Verstegen, et al., J. Reprod. Fert. Suppl., 47:209-218, 1993.

Wakayama et al., Nature, 394:369-374, 1998.

Wakayama et al., Nat Genet., 22:127-128, 1999.

Wells et al., Bio Reprod., 60:996-1005, 1998.

Wilmut et al., Nature, 385:810-813, 1997.

Wood and Wildt, J. Reprod. Fert., 110:355-360, 1997.

WO 94/26884

WO 94/24274

WO 90/03432

WO99/37143A2

WO99/34669A1

WO99/01164A1

What is claimed is:

1. A method of cloning a feline comprising:
(a) fusing an enucleated oocyte and a composition comprising nuclear DNA from a feline somatic cell to form a cybrid; and
(b) transferring the cybrid into the reproductive tract of a female feline, wherein a cloned feline is produced.

2. The method of claim 1, further comprising contacting the enucleated oocyte with the composition prior to fusing.

3. The method of claim 1, wherein the composition comprises a nucleus.

4. The method of claim 3, wherein the nucleus is comprised in a feline somatic cell.

5. The method of claim 1, wherein the cybrid undergoes cell division prior to transferring.

6. The method of claim 5, wherein the cybrid undergoes at least two cell divisions.

7. The method of claim 1, wherein the feline somatic cell is from a cat and the cloned feline is a cat.

8. The method of claim 7, wherein the oocyte and the nuclear DNA are from the same feline species.

9. The method of claim 8, wherein the oocyte and nuclear DNA are from the same feline breed.

10. The method of claim 9, wherein the oocyte and nuclear DNA are from the same animal.

11. The method of claim 1, wherein the oocyte is in metaphase I.

12. The method of claim 1, wherein the oocyte is in metaphase II.

13. The method of claim 1, wherein the oocyte is cultured in vitro prior to or after enucleation.

14. The method of claim 1, further comprising activating the cybrid or the oocyte before the oocyte is fused.

15. The method of claim 14, wherein the cybrid is activated.

16. The method of claim 15, wherein the cybrid is activated upon fusing the oocyte and the composition.

17. The method of claim 14, wherein the oocyte or cybrid is incubated in fusion medium prior to activation.

18. The method of claim 17, wherein the fusion medium lacks Ca²⁺.

19. The method of claim 18, wherein the fusion medium lacks Ca²⁺ and Mg²⁺.

20. The method of claim 15, wherein the cybrid is activated 1 to 8 hours after fusion.

21. The method of claim 14, wherein activating comprises applying an electrical pulse.

22. The method of claim 1, wherein fusing the oocyte and the composition comprises applying an electrical pulse.

23. The method of claim 1, wherein fusing the oocyte and the composition comprises injecting the composition into the oocyte.

24. The method of claim 14, wherein the oocyte or the cybrid is incubated in medium comprising fusion medium during activation.

25. The method of claim 14, wherein the oocyte or cybrid is incubated is fusion medium after activation.

26. The method of claim 25, wherein the fusion medium contains Ca²⁺.

27. The method of claim 1, wherein the cybrid is cultured in vitro for up to 36 hours.

28. The method of claim 27, wherein the cybrid is cultured in vitro overnight.

29. The method of claim 1, wherein the cybrid is frozen and thawed prior to transfer into the female feline.

30. The method of claim 1, wherein the feline somatic cell is an adult cell.

31. The method of claim 30, wherein the adult cell is a cumulus or fibroblast cell.

32. The method of claim 31, wherein the adult cell is a cumulus cell.

33. The method of claim 1, wherein the nuclear DNA comprises a heterologous DNA sequence.

34. The method of claim 1, further comprising administering progesterone to the female feline during pregnancy.

35. A method of cloning a cat comprising:
(a) collecting an oocyte from a first female cat;
(b) incubating the oocyte in vitro in a first medium for 12 to 48 hours;
(c) enucleating the oocyte;
(d) fusing the enucleated oocyte with a composition comprising nuclear DNA from a cat somatic cell in a second medium lacking Ca²⁺ to form a cybrid;
(e) activating the cybrid in a third medium; and
(f) transferring the cybrid into the reproductive tract of a second female cat, wherein a cloned cat is produced.

36. The method of claim 35, wherein the cybrid is activated by applying an electrical current to cybrid in the third medium.

37. The method of claim 35, further comprising incubating the activated cybrid in a fourth medium for up to 7 days prior to transfer into the reproductive tract of the second female cat.

38. The method of claim 37, wherein the fourth medium is IVC 1 medium.

39. The method of claim 38, further comprising incubating the activated cybrid in a fifth medium following incubation in the fourth medium for up to 7 days prior to transfer into the reproductive tract of the second female cat.

40. The method of claim 39, wherein the fifth medium is IVC 2 medium.

41. The method of claim, 39, wherein the activated cybrid is incubated in the fourth and fifth media for a total of up to 7 days prior to transfer into the reproductive tract of the second female cat.

42. The method of claim 35, wherein the third medium comprises fusion medium comprising Ca²⁺.

43. The method of claim 35, further comprising activating the oocyte prior to enucleation.

44. The method of claim 35, further comprising administering progesterone to the second female cat during pregnancy.

45. The method of claim 44, wherein 10 mg of progesterone is administered weekly after 30 days of gestation.

46. A feline whose nuclear genome is identical to a single parent, wherein the parent is multicellular.

47. The feline of claim 46, wherein the parent is a an adult feline.

48. The feline of claim 46, wherein the parent is a feline fetus.

49. The feline of claim 46, wherein the parent is a feline embryo.

50. The feline of claim 46, wherein the feline is a cat.

51. A feline whose nuclear genome is identical to a single adult cell.

52. The feline of claim 46, wherein the cell is a cumulus cell.

53. The feline of claim 46, wherein the cell is a cat cell.

54. A feline whose nuclear genome is identical to a single, multicellular parent produced from the method of claim 1.

55. The feline of claim 54, wherein the feline is a cat.

56. A feline whose nuclear genome is identical to a single, multicellular parent produced from a method comprising:
(a) fusing the oocyte and the composition to form a cybrid;
(b) activating the cybrid; and
(c) transferring the cybrid into the reproductive tract of a female feline, wherein a feline whose nuclear genome is identical to a single, multicellular parent is produced.

57. The feline of claim 56, wherein the oocyte and the nucleus are from the same feline species.

58. The feline of claim 56, wherein the composition comprises a nucleus.

59. The feline of claim 58, wherein the nucleus is comprised in a feline somatic cell.

60. The feline of claim 56, wherein the cybrid undergoes cell division prior to transferring.

61. The feline of claim 56, wherein the oocyte is a feline oocyte.

62. The feline of claim 61, wherein the oocyte and nuclear DNA are from the same breed.

63. The feline of claim 62, wherein the oocyte and nuclear DNA are from the same animal.

64. The feline of claim 56, wherein the oocyte is in metaphase II.

65. The feline of claim 56, wherein the oocyte is cultured in vitro prior to enucleation.

66. The feline of claim 56, wherein activating comprises applying an electrical pulse.

67. The feline of claim 56, wherein fusing the oocyte and the composition comprises applying an electrical pulse.

68. The feline of claim 56, wherein fusing the oocyte and the composition comprises injecting the composition into the oocyte.

69. The feline of claim 56, wherein the oocyte and the composition are fused and activated at the same time.

70. The feline of claim 67, wherein the oocyte and the composition are incubated in media comprising fusion medium.

71. The feline of claim 56, wherein the cybrid is cultured in vitro for up to 36 hours prior to transfer.

72. The feline of claim 71, wherein the cybrid is cultured in vitro for up to 18 hours.

73. The feline of claim 56, wherein the cybrid is cultured in vitro until it has undergone at least one cell division.

74. The feline of claim 73, wherein the cybrid is cultured in vitro until it has undergone at cell division up to 10 times.

75. The feline of claim 56, wherein the estrous cycle of the female feline has been synchronized with the developmental stage of the cybrid.

76. The method of claim 56, wherein the feline is a cat.

77. A progeny feline whose nuclear genome is identical to a single ancestor produced by mating the feline of claim 46 or 56 with a second feline.

78. A progeny feline whose nuclear genome is identical to a single ancestor.

79. A cloned cat produced by the method of claim 35.

80. A progeny cat produced by mating the cloned cat of claim 79 with another cat.

81. A progeny cat whose ancestor is the cat of claim 79.
